(19) United States
(12) Patent Application Publication
(10) Pub. No.: US 2026/0014153 A1
(43) Pub. Date: Jan. 15, 2026

(54) ELACESTRANT IN COMBINATION WITH ABEMACICLIB IN WOMEN WITH BREAST CANCER

(71) Applicant: RADIUS PHARMACEUTICALS, INC., Boston, MA (US)

(72) Inventors: Nina K. ARAGAM, Cambridge, MA (US); Charles MORRIS, Garnet Valley, PA (US)

(21) Appl. No.: 19/338,117

(22) Filed: Sep. 24, 2025

Related U.S. Application Data
(63) Continuation of application No. 19/197,091, filed on May 2, 2025.
Application No. 19/197,091 is a continuation of application No. 17/298,198, filed on May 28, 2021, now abandoned.
(60) Provisional application No. 62/773,960, filed on Nov. 30, 2018.

(51) Int. Cl. A61K 31/506; A61K 31/137; A61P 35/00

(52) U.S. Cl. CPC A61K 31/506; A61K 31/137; A61P 35/00

(57) ABSTRACT

The present disclosure relates to methods of treating breast cancer in a patient, comprising administering to the patient a therapeutic combination comprising elacestrant, or a pharmaceutically acceptable salt thereof, and abemaciclib, or a pharmaceutically acceptable salt thereof. The present disclosure also relates to methods of treating breast cancer in a patient that produce a longer Progression Free Survival time as compared to other treatments.

### CROSS REFERENCE TO RELATED APPLICATIONS

This application claims priority under 35 U.S.C. § 119(e) to U.S. Provisional Patent Application No. 62/773,960, filed Nov. 30, 2018. The entire contents of the aforementioned application are hereby incorporated by reference in its entirety, including drawings.

### TECHNICAL FIELD OF THE INVENTION

The present disclosure relates to methods of treating breast cancer in a patient, comprising administering to the patient a therapeutic combination comprising elacestrant, or a pharmaceutically acceptable salt thereof, and abemaciclib, or a pharmaceutically acceptable salt thereof. The present disclosure also relates to methods of treating breast cancer in a patient that produce a longer Progression Free Survival time as compared to other treatments.

### BACKGROUND OF THE INVENTION

Resistance to endocrine therapy is a challenging aspect in the management of patients with estrogen receptor positive (ER+) breast cancer. Recent studies have demonstrated that acquired resistance can develop after treatment with aromatase inhibitors through the emergence of mutations in the estrogen receptor 1 (ESR1) gene. Another mechanism associated with de novo and acquired resistance is the adaptive upregulation of parallel growth-factor signaling pathways as well as crosstalk between these pathways, including those that promote expression of cyclin D1 and activation of Cyclin Dependent Kinase 4 (CDK4) and CDK6 (CDK4/6).

Strategies designed to disrupt these mechanisms include combining inhibitors of the CDK4/6 pathway with endocrine therapies. Synergistic effects were observed when such combination therapies were evaluated in preclinical studies using endocrine-treatment-naïve and endocrine-treatment-resistant breast cancer cell lines. Furthermore, in the MONARCH-2 clinical trial, the combination of abemaciclib (CDK4/6 inhibitor; Verzenio®, Eli Lilly and Company) and fulvestrant (the only approved selective ER degrader [SERD]; Faslodex®, AstraZeneca) demonstrated improved progression free survival (PFS) compared with fulvestrant alone in patients with ER+, human epidermal growth factor receptor 2-negative (HER2−) metastatic breast cancer.

Elacestrant (RAD1901) is a novel, orally bioavailable SERD. Preclinical data have demonstrated elacestrant, as a single agent and in combination with a CDK4/6 inhibitor, is effective in inhibiting tumor growth in models of ER+ breast cancer with both wild-type and mutant ESR1. Elacestrant monotherapy demonstrated antitumor activity in patient-derived xenograft models of ER+ breast cancer, including those that were insensitive to fulvestrant, estrogen-independent, and/or harbored ESR1 gene mutations. In multiple xenograft models of ER+ breast cancer, the combination of elacestrant with a CDK4/6 inhibitor showed anti-tumor activity that was greater than that observed with either drug alone.

### BRIEF DESCRIPTION OF THE FIGURES

FIG. 1 is a schematic diagram of a study design disclosed herein.

### SUMMARY OF THE INVENTION

In one aspect, the invention relates to a method of treating breast cancer in a patient, comprising administering to the patient a therapeutic combination comprising elacestrant, or a pharmaceutically acceptable salt thereof, and abemaciclib, or a pharmaceutically acceptable salt thereof.

In one embodiment of this aspect, the patient experiences a greater Progression Free Survival time as compared to a patient who was administered a combination of letrozole and abemaciclib, a combination of anastrozole and abemaciclib, or a combination of fulvestrant and abemaciclib.

In another embodiment of this aspect, the patient experiences a greater Progression Free Survival time as compared to a patient who was administered abemaciclib as a monotherapy.

### DETAILED DESCRIPTION OF THE INVENTION

As used herein, RAD1901 or “elacestrant” has the following structure:

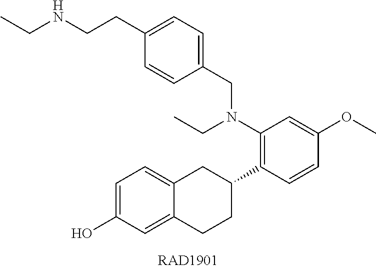 [SMILES: CCNCCc1ccc(CN(CC)c2cc(OC)ccc2[C@@H]2CCc3cc(O)ccc3C2)cc1]

including salts, solvates (e.g. hydrate), and prodrugs thereof.

In some embodiments described herein, RAD1901 is administered as the bis hydrochloride (·2HCl) salt.

As used herein, “abemaciclib” has the following structure:

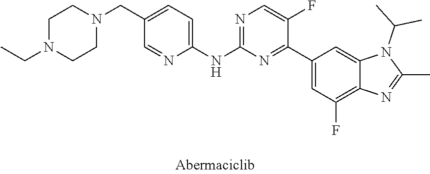 [SMILES: CCN1CCN(Cc2ccc(Nc3ncc(F)c(-c4cc(F)c5nc(C)n(C(C)C)c5c4)n3)nc2)CC1]

including salts, solvates (e.g. hydrate), and prodrugs thereof.

### Definitions

As used herein, the following definitions shall apply unless otherwise indicated.

As used herein, the terms “RAD1901” and “elacestrant” refer to the same chemical compound and are used interchangeably.

“Inhibiting growth” of an ERα-positive tumor as used herein may refer to slowing the rate of tumor growth, or halting tumor growth entirely.

“Tumor regression” or “regression” of an ERα-positive tumor as used herein may refer to reducing the maximum size of a tumor. In certain embodiments, administration of a combination as described herein, or solvates (e.g., hydrate) or salts thereof may result in a decrease in tumor size versus baseline (i.e., size prior to initiation of treatment), or even eradication or partial eradication of a tumor. Accordingly, in certain embodiments the methods of tumor regression provided herein may be alternatively characterized as methods of reducing tumor size versus baseline.

“Tumor” as used herein is a malignant tumor, and is used interchangeably with “cancer.”

“Estrogen receptor alpha” or “ERα” as used herein refers to a polypeptide comprising, consisting of, or consisting essentially of the wild-type ERα amino acid sequence, which is encoded by the gene ESR1.

A tumor that is “positive for estrogen receptor alpha,” “ERα-positive,” “ER+,” or “ERα+” as used herein refers to a tumor in which one or more cells express at least one isoform of ERα.

### Embodiments

In one aspect, the invention relates to a method of treating breast cancer in a patient, comprising administering to the patient a therapeutic combination comprising elacestrant, or a pharmaceutically acceptable salt thereof, and abemaciclib, or a pharmaceutically acceptable salt thereof.

In one embodiment, the patient is a postmenopausal women.

In another embodiment, the patient has not received prior therapy with a CDK4/6 inhibitor or a SERD.

In another embodiment, the breast cancer in the patient has progressed on prior endocrine therapy.

In another embodiment, the breast cancer in the patient is ER+ breast cancer.

The method of claim 5, wherein the breast cancer in the patient is HER2− breast cancer.

In another embodiment, the breast cancer in the patient is advanced or metastatic breast cancer.

In one embodiment, the elacestrant is administered to the patient at a dose of from 200-500 mg/day.

In a further embodiment, the elacestrant is administered to the patient at a dose of from 250-450 mg/day.

In a further embodiment, the elacestrant is administered to the patient at a dose of about 300 mg/day.

In still a further embodiment, the elacestrant is administered to the patient at a dose of about 300 mg/day, in one administration per day.

In another further embodiment, the elacestrant is administered to the patient at a dose of about 400 mg/day.

In still a further embodiment, the elacestrant is administered to the patient at a dose of about 400 mg/day, in one administration per day.

In one embodiment, the abemaciclib is administered to the patient at a dose of from 150-400 mg/day.

In a further embodiment, the abemaciclib is administered to the patient at a dose of about 200 mg/day.

In still a further embodiment, the abemaciclib is administered to the patient at a dose of about 200 mg/day, in two administrations per day.

In another further embodiment, the abemaciclib is administered to the patient at a dose of about 100 mg twice per day.

In one embodiment, the abemaciclib is administered to the patient at a dose of about 300 mg/day.

In a further embodiment, the abemaciclib is administered to the patient at a dose of about 300 mg/day, in two administrations per day.

In still a further embodiment, the abemaciclib is administered to the patient at a dose of about 150 mg twice per day.

In one embodiment, the elacestrant is administered to the patient at a dose of about 400 mg/day, and the abemaciclib is administered to the patient at a dose of about 300 mg/day.

In a further embodiment, the abemaciclib is administered to the patient at a dose of 150 mg twice per day.

In another embodiment, the elacestrant is administered to the patient at a dose of about 300 mg/day, and the abemaciclib is administered to the patient at a dose of about 300 mg/day.

In a further embodiment, the abemaciclib is administered to the patient at a dose of 150 mg twice per day.

In another embodiment, the elacestrant is administered to the patient at a dose of about 300 mg/day, and the abemaciclib is administered to the patient at a dose of about 200 mg/day.

In a further embodiment, the abemaciclib is administered to the patient at a dose of 100 mg twice per day.

In one embodiment, the elacestrant is administered to the patient at a dose that is the maximum tolerated dose for the patient.

In one embodiment, the abemaciclib is administered to the patient at a dose that is the maximum tolerated dose for the patient.

In one embodiment of this aspect, the patient experiences a greater Progression Free Survival time as compared to a patient who was administered a combination of letrozole and abemaciclib, a combination of anastrozole and abemaciclib, or a combination of fulvestrant and abemaciclib.

In a further embodiment, the breast cancer is ER+/HER2− advanced or metastatic breast cancer, and the patient has progressed on or after prior adjuvant or metastatic endocrine therapy, and has not received prior treatment with a CDK4/6 inhibitor or a SERD.

In one embodiment, the patient who was administered a combination of letrozole and abemaciclib was administered 2.5 mg once per day of letrozole and 125 mg twice daily of abemaciclib.

In another embodiment, the patient who was administered a combination of anastrozole and abemaciclib was administered 1 mg once per day of anastrozole and 125 mg twice daily of abemaciclib.

In another embodiment, the patient who was administered a combination of fulvestrant and abemaciclib was administered 500 mg fulvestrant injection as two 5 mL injections intramuscularly into the buttocks (gluteal area), at a rate of 1-2 minutes per injection, one in each buttock, on days 1, 15, and 29 and once monthly thereafter, and 125 mg twice daily of abemaciclib.

In another embodiment, the patient experiences a greater Progression Free Survival time as compared to a patient who was administered a combination of letrozole and abemaciclib and wherein the breast cancer is ER+/HER2− advanced or metastatic breast cancer, and the patient has not received prior systemic anti-cancer therapies for their advanced/metastatic disease, and has not received prior treatment with a CDK4/6 inhibitor or a SERD.

In a further embodiment, the patient who was administered a combination of letrozole and abemaciclib was administered 2.5 mg once per day of letrozole and 125 mg twice daily of abemaciclib.

In another embodiment of this aspect, the patient experiences a greater Progression Free Survival time as compared to a patient who was administered abemaciclib as a monotherapy.

In a further embodiment, the breast cancer is ER+/HER2− advanced or metastatic breast cancer, and the patient has received prior systemic anti-cancer therapy including ≤2 prior chemotherapy for metastatic breast cancer permitted for their advanced or metastatic disease, and wherein the prior systemic anti-cancer therapy did not include a CDK4/6 inhibitor or a SERD.

In still a further embodiment, the patient who was administered abemaciclib as a monotherapy was administered 200 mg of abemaciclib twice daily.

### Formulations, Administrations, and Uses

Combination therapy comprising elacestrant and a CDK inhibitor has been previously described in U.S. Patent Application Publication number 2018/0169101, the entire contents of which is hereby incorporated by reference in its entirety.

Combination of RAD1901 or solvates (e.g., hydrate) or salts thereof and abemaciclib.

Both RAD1901 or solvates (e.g., hydrate) or salts thereof and abemaciclib, when administered alone to a subject, have a therapeutic effect on one or more cancers or tumors. It was surprisingly discovered that when administered in combination to a subject, RAD1901 or solvates (e.g., hydrate) or salts thereof and abemaciclib have a significantly improved effect on the cancers/tumors.

Tumor growth inhibition or regression may be localized to a single tumor or to a set of tumors within a specific tissue or organ, or may be systemic (i.e., affecting tumors in all tissues or organs).

As RAD1901 is known to preferentially bind ERα versus estrogen receptor beta (ERβ), unless specified otherwise, estrogen receptor, estrogen receptor alpha, ERα, ER, wild-type ERα, and ESR1 are used interchangeably herein. In certain embodiments, ER+ cells overexpress ERα. In certain embodiments, the patient has one or more cells within the tumor expressing one or more forms of ERβ. In certain embodiments, the ERα-positive tumor and/or cancer is associated with breast, uterine, ovarian, or pituitary cancer. In certain of these embodiments, the patient has a tumor located in breast, uterine, ovarian, or pituitary tissue. In those embodiments where the patient has a tumor located in the breast, the tumor may be associated with luminal breast cancer that may or may not be positive for HER2, and for HER2+ tumors, the tumors may express high or low HER2. In other embodiments, the patient has a tumor located in another tissue or organ (e.g., bone, muscle, brain), but is nonetheless associated with breast, uterine, ovarian, or pituitary cancer (e.g., tumors derived from migration or metastasis of breast, uterine, ovarian, or pituitary cancer). Accordingly, in certain embodiments of the tumor growth inhibition or tumor regression methods provided herein, the tumor being targeted is a metastatic tumor and/or the tumor has an overexpression of ER in other organs (e.g., bones and/or muscles). In certain embodiments, the tumor being targeted is a brain tumor and/or cancer. In certain embodiments, the tumor being targeted is more sensitive to a treatment of RAD1901 and abemaciclib than treatment with another SERD (e.g., fulvestrant, TAS-108 (SR16234), ZK191703, RU58668, GDC-0810 (ARN-810), GW5638/DPC974, SRN-927, ICI182782 and AZD9496), Her2 inhibitors (e.g., trastuzumab, lapatinib, ado-trastuzumab emtansine, and/or pertuzumab), chemo therapy (e.g., abraxane, adriamycin, carboplatin, cytoxan, daunorubicin, doxil, ellence, fluorouracil, gemzar, helaven, Ixempra, methotrexate, mitomycin, micoxantrone, navelbine, taxol, taxotere, thiotepa, vincristine, and xeloda), aromatase inhibitor (e.g., anastrozole, exemestane, and letrozole), selective estrogen receptor modulators (e.g., tamoxifen, raloxifene, lasofoxifene, and/or toremifene), angiogenesis inhibitor (e.g., bevacizumab), and/or rituximab.

In certain embodiments of the tumor growth inhibition or tumor regression methods provided herein, the methods further comprise a step of determining whether a patient has a tumor expressing ERα prior to administering a combination of abemaciclib and RAD1901 or solvates (e.g., hydrate) or salts thereof. In certain embodiments of the tumor growth inhibition or tumor regression methods provided herein, the methods further comprise a step of determining whether the patient has a tumor expressing mutant ERα prior to administering a combination of abemaciclib and RAD1901 or solvates (e.g., hydrate) or salts thereof. In certain embodiments of the tumor growth inhibition or tumor regression methods provided herein, the methods further comprise a step of determining whether a patient has a tumor expressing ERα that is responsive or non-responsive to fulvestrant treatment prior to administering a combination of abemaciclib and RAD1901 or solvates (e.g., hydrate) or salts thereof. These determinations may be made using any method of expression detection known in the art, and may be performed in vitro using a tumor or tissue sample removed from the subject.

In addition to demonstrating the ability of RAD1901 to inhibit tumor growth in tumors expressing wild-type ERα, RAD1901 exhibits the unexpected ability to inhibit the growth of tumors expressing a mutant form of ERα, namely Y537S ERα. Computer modeling evaluations of examples of ERα mutations showed that none of these mutations were expected to impact the LBD or specifically hinder RAD1901 binding, e.g., ERα having one or more mutants selected from the group consisting of ERα with Y537X mutant wherein X is S, N, or C, ERα with D538G mutant, and ERα with S463P mutant. Based on these results, methods are provided herein for inhibiting growth or producing regression of a tumor that is positive for ERα having one or more mutants within the ligand-binding domain (LBD), selected from the group consisting of Y537X1 wherein X1 is S, N, or C, D538G, L536X2 wherein X2 is R or Q, P535H, V534E, S463P, V392I, E380Q, especially Y537S ERα, in a subject with cancer by administering to the subject a therapeutically effective amount of a combination of abemaciclib and RAD1901 or solvates (e.g., hydrate) or salts thereof. In certain embodiments, RAD1901 or solvates (e.g., hydrate) or salts thereof. “Mutant ERα” as used herein refers to ERα comprising one or more substitutions or deletions, and variants thereof comprising, consisting of, or consisting essentially of an amino acid sequence with at least 80%, at least 85%, at least 90%, at least 95%, at least 97%, at least 98%, at least 99%, or at least 99.5% identity to the amino acid sequence of ERα.

In addition to inhibiting breast cancer tumor growth in an animal xenograft model, RAD1901 exhibits significant accumulation within tumor cells, and is capable of penetrating the blood-brain barrier. The ability to penetrate the blood-brain barrier was confirmed by showing that RAD1901 administration significantly prolonged survival in a brain metastasis xenograft model. Accordingly, in certain embodiments of the tumor growth inhibition or tumor regression methods provided herein, the ERα-positive tumor being targeted is located in the brain or elsewhere in the central nervous system. In certain of these embodiments, the ERα-positive tumor is primarily associated with brain cancer. In other embodiments, the ERα-positive tumor is a metastatic tumor that is primarily associated with another type of cancer, such as breast, uterine, ovarian, or pituitary cancer, or a tumor that has migrated from another tissue or organ. In certain of these embodiments, the tumor is a brain metastases, such as breast cancer brain metastases (BCBM). In certain embodiments of the methods disclosed herein, RAD1901 or solvates (e.g., hydrate) or salts thereof accumulate in one or more cells within a target tumor.

In certain embodiments of the methods disclosed herein, RAD1901 or solvates (e.g., hydrate) or salts thereof preferably accumulate in tumor at a T/P (RAD1901 concentration in tumor/RAD1901 concentration in plasma) ratio of about 15 or higher, about 18 or higher, about 19 or higher, about 20 or higher, about 25 or higher, about 28 or higher, about 30 or higher, about 33 or higher, about 35 or higher, or about 40 or higher.

Results have shown that RAD1901 administration protects against bone loss in ovariectomized rats. Accordingly, in certain embodiments of the tumor growth inhibition or tumor regression methods provided herein, administration of a combination of abemaciclib and RAD1901 or solvates (e.g., hydrate) or salts thereof does not have undesirable effects on bone, including for example undesirable effects on bone volume density, bone surface density, bone mineral density, trabecular number, trabecular thickness, trabecular spacing, connectivity density, and/or apparent bone density of the treated subject. As tamoxifen may be associated with bone loss in premenopausal women, and fulvestrant may impair the bone structures due to its mechanism of action, a combination of abemaciclib and RAD1901 or solvates (e.g., hydrate) or salts thereof can be particularly useful for premenopausal women, tumors resistant to tamoxifen or antiestrogen therapy, and patients having osteoporosis and/or high risk of osteoporosis.

It has been shown that RAD1901 antagonized estradiol stimulation of uterine tissues in ovariectomized rats. Furthermore, in human subjects treated with RAD1901 at a dosage of 200 mg or up to 500 mg q.d., standardized uptake value (SUV) for uterus, muscle, and bone tissues that did not significantly express ER showed hardly any changes in signals pre- and post-treatment. Accordingly, in certain embodiments, such administration also does not result in undesirable effects on other tissues, including for example uterine, muscle, or breast tissue.

RAD1901 or solvates (e.g., hydrate) or salts thereof and abemaciclib are administered in combination to a subject in need. The phrase “in combination” means RAD1901 or solvates (e.g., hydrate) or salts thereof may be administered before, during, or after the administration of abemaciclib. For example, RAD1901 or solvates (e.g., hydrate) or salts thereof and abemaciclib can be administered in about one week apart, about 6 days apart, about 5 days apart, about 4 days apart, about 3 days apart, about 2 days apart, about 24 hours apart, about 23 hours apart, about 22 hours apart, about 21 hours apart, about 20 hours apart, about 19 hours apart, about 18 hours apart, about 17 hours apart, about 16 hours apart, about 15 hours apart, about 14 hours apart, about 13 hours apart, about 12 hours apart, about 11 hours apart, about 10 hours apart, about 9 hours apart, about 8 hours apart, about 7 hours apart, about 6 hours apart, about 5 hours apart, about 4 hours apart, about 3 hours apart, about 2 hours apart, about 1 hour apart, about 55 minutes apart, about 50 minutes apart, about 45 minutes apart, about 40 minutes apart, about 35 minutes apart, about 30 minutes apart, about 25 minutes apart, about 20 minutes apart, about 15 minutes apart, about 10 minutes apart, or about 5 minutes apart. In other embodiments RAD1901 or solvates (e.g., hydrate) or salts thereof and abemaciclib are administered to the subject simultaneously or substantially simultaneously. In certain of these embodiments, RAD1901 or solvates (e.g., hydrate) or salts thereof and abemaciclib may be administered as part of a single formulation.

### Dosage

A therapeutically effective amount of a combination of abemaciclib and RAD1901 or solvates (e.g., hydrate) or salts thereof for use in the methods disclosed herein is an amount that, when administered over a particular time interval, results in achievement of one or more therapeutic benchmarks (e.g., slowing or halting of tumor growth, resulting in tumor regression, cessation of symptoms, etc.). The combination for use in the presently disclosed methods may be administered to a subject one time or multiple times. In those embodiments wherein the compounds are administered multiple times, they may be administered at a set interval, e.g., daily, every other day, weekly, or monthly. Alternatively, they can be administered at an irregular interval, for example on an as-needed basis based on symptoms, patient health, and the like. A therapeutically effective amount of the combination may be administered q.d. for one day, at least 2 days, at least 3 days, at least 4 days, at least 5 days, at least 6 days, at least 7 days, at least 10 days, or at least 15 days. Optionally, the status of the cancer or the regression of the tumor is monitored during or after the treatment, for example, by a FES-PET scan of the subject. The dosage of the combination administered to the subject can be increased or decreased depending on the status of the cancer or the regression of the tumor detected.

Ideally, the therapeutically effective amount does not exceed the maximum tolerated dosage at which 50% or more of treated subjects experience nausea or other toxicity reactions that prevent further drug administrations. A therapeutically effective amount may vary for a subject depending on a variety of factors, including variety and extent of the symptoms, sex, age, body weight, or general health of the subject, administration mode and salt or solvate type, variation in susceptibility to the drug, the specific type of the disease, and the like.

Examples of therapeutically effective amounts of a RAD1901 or solvates (e.g., hydrate) or salts thereof for use in the methods disclosed herein include, without limitation, about 150 to about 1,500 mg, about 200 to about 1,500 mg, about 250 to about 1,500 mg, or about 300 to about 1,500 mg dosage q.d. for subjects having resistant ER-driven tumors or cancers; about 150 to about 1,500 mg, about 200 to about 1,000 mg or about 250 to about 1,000 mg or about 300 to about 1,000 mg dosage q.d. for subjects having both wild-type ER driven tumors and/or cancers and resistant tumors and/or cancers; and about 300 to about 500 mg, about 300 to about 550 mg, about 300 to about 600 mg, about 250 to about 500 mg, about 250 to about 550 mg, about 250 to about 600 mg, about 200 to about 500 mg, about 200 to about 550 mg, about 200 to about 600 mg, about 150 to about 500 mg, about 150 to about 550 mg, or about 150 to about 600 mg q.d. dosage for subjects having majorly wild-type ER driven tumors and/or cancers. In certain embodiments, the dosage of a compound of Formula I (e.g., RAD1901) or a salt or solvate thereof for use in the presently disclosed methods general for an adult subject may be approximately 200 mg, 400 mg, 30 mg to 2,000 mg, 100 mg to 1,500 mg, or 150 mg to 1,500 mg p.o., q.d. This daily dosage may be achieved via a single administration or multiple administrations.

Dosing of RAD1901 with abemaciclib can be accomplished with RAD1901 at 100, 200, 300, 400, 500, 600, 700, 800, 900 or 1,000 mg per day. In particular, 200 mg, 400 mg, 500 mg, 600 mg, 800 mg and 1,000 mg per day are noted. Under certain circumstances a BID dosing schedule is preferred. The surprisingly long half life of RAD1901 in humans after PO dosing make this option particularly viable. Accordingly, the drug may be administered as 200 mg bid (400 mg total daily), 250 mg bid (500 mg total daily), 300 mg bid (600 mg total daily), 400 mg bid (800 mg daily) or 500 mg bid (1,000 mg total daily). Preferably the dosing is oral. The dose of abemaciclib may be 50 mg to 500 mg daily, or 150 mg to 450 mg daily and the dosing can be daily in 28 day cycles or less than 28 days per 28 day cycles such as 21 days per 28 day cycle or 14 days per 28 day cycle or 7 days per 28 day cycles. In some embodiments, the abemaciclib is dosed once daily or preferably on a bid schedule where dosing is oral. In the case of bid dosing, the doses can be separated by 4 hours, 8 hours or 12 hours. In certain embodiments, the abemaciclib is dosed at 150 mg bid via oral where the doses are recommended to be spaced out by 12 hours.

As has been discovered, there appears to be a remarkable synergy between RAD1901 and cdk 4/6 inhibitors and therefore, a dose reduction of RAD1901 and/or abemaciclib from the usual recommended or approved dose is contemplated. For example, RAD1901 may be recommended for monotherapy treatment at doses of 100, 200, 300, 400, 500, 600, 700, 800, 900 or 1,000 mg or more specifically at 200 mg, 400 mg, 500 mg, 600 mg, 800 mg and 1,000 mg per day. In combination, a reduction of the specified dose by a given fraction means that doses of 25% to 75% less than the usual dose are possible. By way of non-limiting example, a recommended dose of RAD1901 of 400 mg per day may be reduced to between a final dose of 100 mg and 300 mg per day, or 100 mg per day, 200 mg per day or 300 mg per day. If the RAD1901 dose is reduced as described, the same percent reduction is generally applied whether the dosing is bid or once daily. For example, a 400 mg bid dose reduced by 50% would be administered on a 200 mg bid schedule. In some exceptions, a reduction of a daily recommended bid dose may be sufficient to allow for the total daily dose to be administered as a once daily dose. For example, a normal bid dose of 300 mg that is given in combination with abemaciclib may be reduced by 50%. Accordingly, the dose may be given as 150 mg bid or 300 mg once daily.

Similarly, the normal recommended dose of abemaciclib may be reduced when used in combination with RAD1901. The dose of abemaciclib may be reduced and combined with the normal recommended monotherapy dose of RAD1901 or a reduced RAD1901 dose wherein the reduced dose is 25% to 75% less than the normal recommended dose as exemplified immediately above. For example, a recommended dose of abemaciclib of 150 mg bid might be given as a bid dose of 25% to 75% less than the 150 mg bid dose. For example, 150 mg bid of abemaciclib may be reduced to a bid dose of 37.5 mg to 112.5 mg (total daily dose of 75 mg to 225 mg). Alternatively, it may be desirable to reduce the frequency of abemaciclib from a recommended 28 day on cycle to some amount less. For example, the dosing frequency can be reduced to 22 days to 27 days out of a 28 day cycle or to 21 days out of a 28 day cycle, or the dosing frequency may be reduced to 15 days to 20 days out of a 28 day cycle or to 14 days out of a 28 day cycle, or the dosing frequency may be reduced to 8 days to 13 days out of a 28 day cycle or to just 7 days out of a 28 day cycle. The days dosed may be consecutive or combined as needed under the circumstance. In one embodiment, the total dose over a dosing interval is reduced by 25% to 75% of the recommended dose and that reduction may come as a result of less frequent dosing, reduced dosage or a combination thereof. For example, a recommended dosing cycle of 28 days of abemaciclib at a dose of 150 mg bid (300 mg total daily) results in a total dose over 28 days of 8,400 mg (28 days times 300 mg total per day). This amount can be reduced to between from 2,100 mg per 28 day to 6,300 mg per 28 day.

In certain embodiments, a therapeutically effective amount of the combination may utilize a therapeutically effective amount of either compound administered alone. In other embodiments, due to the significantly improved, synergistic therapeutic effect achieved by the combination, the therapeutically effective amounts of RAD1901 or solvates (e.g., hydrate) or salts thereof and abemaciclib when administered in the combination may be smaller than the therapeutically effective amounts of RAD1901 or solvates (e.g., hydrate) or salts thereof and abemaciclib required when administered alone; and one or both compounds may be administered at a dosage that is lower than the dosage at which they would normally be administered when given separately. Without being bound by any specific theory, the combination therapy achieves a significantly improved effect by reducing the dosage of at least one or all of RAD1901 or solvates (e.g., hydrate) or salts thereof and abemaciclib, thereby eliminating or alleviating undesirable toxic side effects.

In some embodiments, the therapeutically effective amount of RAD1901 or solvates (e.g., hydrate) or salts thereof when administered as part of the combination is about 30% to about 200%, about 40% to about 200%, about 50% to about 200%, about 60% to about 200%, about 70% to about 200%, about 80% to about 200%, about 90% to about 200%, about 100% to about 200%, 30% to about 150%, about 40% to about 150%, about 50% to about 150%, about 60% to about 150%, about 70% to about 150%, about 80% to about 150%, about 90% to about 150%, about 100% to about 150%, about 30% to about 120%, about 40% to about 120%, about 50% to about 120%, about 60% to about 120%, about 70% to about 120%, about 80% to about 120%, about 90% to about 120%, about 100% to about 120%, 30% to about 110%, about 40% to about 110%, about 50% to about 110%, about 60% to about 110%, about 70% to about 110%, about 80% to about 110%, about 90% to about 110%, or about 100% to about 110% of the therapeutically effective amount of RAD1901 or solvates (e.g., hydrate) or salts thereof when administered alone. In some embodiments, the therapeutically effective amount of abemaciclib when administered as part of the combination is about 30% to about 200%, about 40% to about 200%, about 50% to about 200%, about 60% to about 200%, about 70% to about 200%, about 80% to about 200%, about 90% to about 200%, about 100% to about 200%, 30% to about 150%, about 40% to about 150%, about 50% to about 150%, about 60% to about 150%, about 70% to about 150%, about 80% to about 150%, about 90% to about 150%, about 100% to about 150%, about 30% to about 120%, about 40% to about 120%, about 50% to about 120%, about 60% to about 120%, about 70% to about 120%, about 80% to about 120%, about 90% to about 120%, about 100% to about 120%, 30% to about 110%, about 40% to about 110%, about 50% to about 110%, about 60% to about 110%, about 70% to about 110%, about 80% to about 110%, about 90% to about 110%, or about 100% to about 110% of the therapeutically effective amount of abemaciclib when administered alone.

In certain embodiments, the cancers or tumors are resistant ER-driven cancers or tumors (e.g. having mutant ER binding domains (e.g. ERα comprising one or more mutations including, but not limited to, Y537X1 wherein X1 is S, N, or C, D538G, L536X2 wherein X2 is R or Q, P535H, V534E, S463P, V392I, E380Q and combinations thereof), overexpressors of the ERs or tumor and/or cancer proliferation becomes ligand independent, or tumors and/or cancers that progress with treatment of another SERD (e.g., fulvestrant, TAS-108 (SR16234), ZK191703, RU58668, GDC-0810 (ARN-810), GW5638/DPC974, SRN-927, ICI182782 and AZD9496), Her2 inhibitors (e.g., trastuzumab, lapatinib, ado-trastuzumab emtansine, and/or pertuzumab), chemo therapy (e.g., abraxane, adriamycin, carboplatin, cytoxan, daunorubicin, doxil, ellence, fluorouracil, gemzar, helaven, lxempra, methotrexate, mitomycin, micoxantrone, navelbine, taxol, taxotere, thiotepa, vincristine, and xeloda), aromatase inhibitor (e.g., anastrozole, exemestane, and letrozole), selective estrogen receptor modulators (e.g., tamoxifen, raloxifene, lasofoxifene, and/or toremifene), angiogenesis inhibitor (e.g., bevacizumab), and/or rituximab.

In certain embodiments, the dosage of RAD1901 or solvates (e.g., hydrate) or salts thereof in a combination with abemaciclib for use in the presently disclosed methods general for an adult subject may be approximately 30 mg to 2,000 mg, 100 mg to 1,500 mg, or 150 mg to 1,500 mg p.o., q.d. This daily dosage may be achieved via a single administration or multiple administrations.

A combination of abemaciclib and RAD1901 or solvates (e.g., hydrate) or salts thereof may be administered to a subject one time or multiple times. In those embodiments wherein the compounds are administered multiple times, they may be administered at a set interval, e.g., daily, every other day, weekly, or monthly. Alternatively, they can be administered at an irregular interval, for example on an as-needed basis based on symptoms, patient health, and the like.

### Formulation

In some embodiments, RAD1901 or solvates (e.g., hydrate) or salts thereof and abemaciclib are administered in separate formulations. In certain of these embodiments, the formulations may be of the same type. For example, both formulations may be designed for oral administration (e.g., via two separate pills) or for injection (e.g., via two separate injectable formulations). In other embodiments, RAD1901 or solvates (e.g., hydrate) or salts thereof and abemaciclib may be formulated in different types of formulations. For example, one compound may be in a formulation designed for oral administration, while the other is in a formulation designed for injection.

In other embodiments, RAD1901 or solvates (e.g., hydrate) or salts thereof and abemaciclib are administered as part of a single formulation. For example, RAD1901 or solvates (e.g., hydrate) or salts thereof and abemaciclib are formulated in a single pill for oral administration or in a single dose for injection. Provided herein in certain embodiments are combination formulations comprising RAD1901 or solvates (e.g., hydrate) or salts thereof and abemaciclib. In certain embodiments, administration of the compounds in a single formulation improves patient compliance.

The therapeutically effective amount of each compound when administered in combination may be lower than the therapeutically effective amount of each compound administered alone.

In some embodiments, a formulation comprising RAD1901 or solvates (e.g., hydrate) or salts thereof, abemaciclib, or both RAD1901 or solvates (e.g., hydrate) or salts thereof and the abemaciclib may further comprise one or more pharmaceutical excipients, carriers, adjuvants, and/or preservatives.

The RAD1901 or solvates (e.g., hydrate) or salts thereof and abemaciclib for use in the presently disclosed methods can be formulated into unit dosage forms, meaning physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times q.d.). When multiple daily doses are used, the unit dosage form can be the same or different for each dose. In certain embodiments, the compounds may be formulated for controlled release.

The RAD1901 or solvates (e.g., hydrate) or salts thereof and salts or solvates and abemaciclib for use in the presently disclosed methods can be formulated according to any available conventional method. Examples of preferred dosage forms include a tablet, a powder, a subtle granule, a granule, a coated tablet, a capsule, a syrup, a troche, an inhalant, a suppository, an injectable, an ointment, an ophthalmic ointment, an eye drop, a nasal drop, an ear drop, a cataplasm, a lotion and the like. In the formulation, generally used additives such as a diluent, a binder, an disintegrant, a lubricant, a colorant, a flavoring agent, and if necessary, a stabilizer, an emulsifier, an absorption enhancer, a surfactant, a pH adjuster, an antiseptic, an antioxidant and the like can be used. In addition, the formulation is also carried out by combining compositions that are generally used as a raw material for pharmaceutical formulation, according to the conventional methods. Examples of these compositions include, for example, (1) an oil such as a soybean oil, a beef tallow and synthetic glyceride; (2) hydrocarbon such as liquid paraffin, squalane and solid paraffin; (3) ester oil such as octyldodecyl myristic acid and isopropyl myristic acid; (4) higher alcohol such as cetostearyl alcohol and behenyl alcohol; (5) a silicon resin; (6) a silicon oil; (7) a surfactant such as polyoxyethylene fatty acid ester, sorbitan fatty acid ester, glycerin fatty acid ester, polyoxyethylene sorbitan fatty acid ester, a solid polyoxyethylene castor oil and polyoxyethylene polyoxypropylene block co-polymer; (8) water soluble macromolecule such as hydroxyethyl cellulose, polyacrylic acid, carboxyvinyl polymer, polyethyleneglycol, polyvinylpyrrolidone and methylcellulose; (9) lower alcohol such as ethanol and isopropanol; (10) multivalent alcohol such as glycerin, propyleneglycol, dipropyleneglycol and sorbitol; (11) a sugar such as glucose and cane sugar; (12) an inorganic powder such as anhydrous silicic acid, aluminum magnesium silicate and aluminum silicate; (13) purified water, and the like. Additives for use in the above formulations may include, for example, 1) lactose, corn starch, sucrose, glucose, mannitol, sorbitol, crystalline cellulose and silicon dioxide as the diluent; 2) polyvinyl alcohol, polyvinyl ether, methyl cellulose, ethyl cellulose, gum arabic, tragacanth, gelatine, shellac, hydroxypropyl cellulose, hydroxypropylmethyl cellulose, polyvinylpyrrolidone, polypropylene glycol-poly oxyethylene-block co-polymer, meglumine, calcium citrate, dextrin, pectin and the like as the binder; 3) starch, agar, gelatine powder, crystalline cellulose, calcium carbonate, sodium bicarbonate, calcium citrate, dextrin, pectic, carboxymethylcellulose/calcium and the like as the disintegrant; 4) magnesium stearate, talc, polyethyleneglycol, silica, condensed plant oil and the like as the lubricant; 5) any colorants whose addition is pharmaceutically acceptable is adequate as the colorant; 6) cocoa powder, menthol, aromatizer, peppermint oil, cinnamon powder as the flavoring agent; 7) antioxidants whose addition is pharmaceutically accepted such as ascorbic acid or alpha-tophenol.

Abemaciclib and RAD1901 or solvates (e.g., hydrate) or salts thereof for use in the presently disclosed methods can be formulated into a pharmaceutical composition as any one or more of the active compounds described herein and a physiologically acceptable carrier (also referred to as a pharmaceutically acceptable carrier or solution or diluent). Such carriers and solutions include pharmaceutically acceptable salts and solvates of compounds used in the methods of the instant invention, and mixtures comprising two or more of such compounds, pharmaceutically acceptable salts of the compounds and pharmaceutically acceptable solvates of the compounds. Such compositions are prepared in accordance with acceptable pharmaceutical procedures such as described in Remington's Pharmaceutical Sciences, 17th edition, ed. Alfonso R. Gennaro, Mack Publishing Company, Eaton, Pa. (1985), which is incorporated herein by reference.

The term “pharmaceutically acceptable carrier” refers to a carrier that does not cause an allergic reaction or other untoward effect in patients to whom it is administered and are compatible with the other ingredients in the formulation. Pharmaceutically acceptable carriers include, for example, pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically acceptable carriers may further comprise minor amounts of auxiliary substances such as wetting or emulsifying agents, preservatives or buffers, which enhance the shelf life or effectiveness of the therapeutic agent.

Abemaciclib and RAD1901 or solvates (e.g., hydrate) or salts thereof in a free form can be converted into a salt by conventional methods. The term “salt” used herein is not limited as long as the salt is formed with RAD1901 or solvates (e.g., hydrate) or salts thereof and is pharmacologically acceptable; preferred examples of salts include a hydrohalide salt (for instance, hydrochloride, hydrobromide, hydroiodide and the like), an inorganic acid salt (for instance, sulfate, nitrate, perchlorate, phosphate, carbonate, bicarbonate and the like), an organic carboxylate salt (for instance, acetate salt, maleate salt, tartrate salt, fumarate salt, citrate salt and the like), an organic sulfonate salt (for instance, methanesulfonate salt, ethanesulfonate salt, benzenesulfonate salt, toluenesulfonate salt, camphorsulfonate salt and the like), an amino acid salt (for instance, aspartate salt, glutamate salt and the like), a quaternary ammonium salt, an alkaline metal salt (for instance, sodium salt, potassium salt and the like), an alkaline earth metal salt (magnesium salt, calcium salt and the like) and the like. In addition, hydrochloride salt, sulfate salt, methanesulfonate salt, acetate salt and the like are preferred as “pharmacologically acceptable salt” of the compounds according to the present invention.

Isomers of RAD1901 or solvates (e.g., hydrate) or salts thereof and/or abemaciclib (e.g., geometric isomers, optical isomers, rotamers, tautomers, and the like) can be purified using general separation means, including for example recrystallization, optical resolution such as diastereomeric salt method, enzyme fractionation method, various chromatographies (for instance, thin layer chromatography, column chromatography, glass chromatography and the like) into a single isomer. The term “a single isomer” herein includes not only an isomer having a purity of 100%, but also an isomer containing an isomer other than the target, which exists even through the conventional purification operation. A crystal polymorph sometimes exists for RAD1901 or solvates (e.g., hydrate) or salts thereof and/or abemaciclib, and all crystal polymorphs thereof are included in the present invention. The crystal polymorph is sometimes single and sometimes a mixture, and both are included herein.

In certain embodiments, RAD1901 or solvates (e.g., hydrate) or salts thereof and/or abemaciclib may be in a prodrug form, meaning that it must undergo some alteration (e.g., oxidation or hydrolysis) to achieve its active form. Alternative, RAD1901 or solvates (e.g., hydrate) or salts thereof and/or abemaciclib may be a compound generated by alteration of a parental prodrug to its active form.

### Administration Route

Administration routes of RAD1901 or solvates (e.g., hydrate) or salts thereof and/or abemaciclib include but not limited to topical administration, oral administration, intradermal administration, intramuscular administration, intraperitoneal administration, intravenous administration, intravesical infusion, subcutaneous administration, transdermal administration, and transmucosal administration.

### Gene Profiling

In certain embodiments, the methods of tumor growth inhibition or tumor regression provided herein further comprise gene profiling the subject, wherein the gene to be profiled is one or more genes selected from the group consisting of ABL1, AKT1, AKT2, ALK, APC, AR, ARID1A, ASXL1, ATM, AURKA, BAP, BAP1, BCL2L11, BCR, BRAF, BRCA1, BRCA2, CCND1, CCND2, CCND3, CCNE1, CDH1, CDK4, CDK6, CDK8, CDKNIA, CDKNIB, CDKN2A, CDKN2B, CEBPA, CTNNB1, DDR2, DNMT3A, E2F3, EGFR, EML4, EPHB2, ERBB2, ERBB3, ESR1, EWSR1, FBXW7, FGF4, FGFR1, FGFR2, FGFR3, FLT3, FRS2, HIF1A, HRAS, IDH1, IDH2, IGFIR, JAK2, KDM6A, KDR, KIF5B, KIT, KRAS, LRPIB, MAP2K1, MAP2K4, MCL1, MDM2, MDM4, MET, MGMT, MLL, MPL, MSH6, MTOR, MYC, NF1, NF2, NKX2-1, NOTCH1, NPM, NRAS, PDGFRA, PIK3CA, PIK3R1, PML, PTEN, PTPRD, RARA, RB1, RET, RICTOR, ROSI, RPTOR, RUNX1, SMAD4, SMARCA4, SOX2, STK11, TET2, TP53, TSC1, TSC2, and VHL.

In some embodiments, this invention provides a method of treating a subpopulation of breast cancer patients wherein said sub-population has increased expression of one or more of the genes disclosed supra, and treating said sub-population with an effective dose of a combination of abemaciclib and RAD1901 or solvates (e.g., hydrate) or salts thereof according to the dosing embodiments as described in this disclosure.

### Dose Adjusting

In addition to establishing the ability of RAD1901 to inhibit tumor growth, RAD1901 inhibits estradiol binding to ER in the uterus and pituitary. In these experiments, estradiol binding to ER in uterine and pituitary tissue was evaluated by FES-PET imaging. After treatment with RAD1901, the observed level of ER binding was at or below background levels. These results establish that the antagonistic effect of RAD1901 on ER activity can be evaluated using real-time scanning. Based on these results, methods are provided herein for monitoring the efficacy of treatment RAD1901 or solvates (e.g., hydrate) or salts thereof in a combination therapy disclosed herein by measuring estradiol-ER binding in one or more target tissues, wherein a decrease or disappearance in binding indicates efficacy.

Further provided are methods of adjusting the dosage of RAD1901 or solvates (e.g., hydrate) or salts thereof in a combination therapy disclosed herein based on estradiol-ER binding. In certain embodiments of these methods, binding is measured at some point following one or more administrations of a first dosage of the compound. If estradiol-ER binding is not affected or exhibits a decrease below a predetermined threshold (e.g., a decrease in binding versus baseline of less than 5%, less than 10%, less than 20%, less than 30%, or less than 50%), the first dosage is deemed to be too low. In certain embodiments, these methods comprise an additional step of administering an increased second dosage of the compound. These steps can be repeated, with dosage repeatedly increased until the desired reduction in estradiol-ER binding is achieved. In certain embodiments, these steps can be incorporated into the methods of inhibiting tumor growth provided herein. In these methods, estradiol-ER binding can serve as a proxy for tumor growth inhibition, or a supplemental means of evaluating growth inhibition. In other embodiments, these methods can be used in conjunction with the administration of RAD1901 or solvates (e.g., hydrate) or salts thereof for purposes other than inhibition of tumor growth, including for example inhibition of cancer cell proliferation.

In certain embodiments, the methods provided herein for adjusting the dosage of RAD1901 or salt or solvate (e.g., hydrate) thereof in a combination therapy comprise:
(1) administering a first dosage of RAD1901 or salt or solvate (e.g., hydrate) thereof (e.g., about 350 to about 500 or about 200 to about 600 mg/day) for 3, 4, 5, 6, or 7 days;
(2) detecting estradiol-ER binding activity; wherein:
   (i) if the ER binding activity is not detectable or is below a predetermined threshold level, continuing to administer the first dosage (i.e., maintain the dosage level); or
   (ii) if the ER binding activity is detectable or is above a predetermined threshold level, administering a second dosage that is greater than the first dosage (e.g., the first dosage plus about 50 to about 200 mg) for 3, 4, 5, 6, or 7 days, then proceeding to step (3);
(3) detecting estradiol-ER binding activity; wherein
   (i) if the ER binding activity is not detectable or is below a predetermined threshold level, continuing to administer the second dosage (i.e., maintain the dosage level); or
   (ii) if the ER binding activity is detectable or is above a predetermined threshold level, administering a third dosage that is greater than the second dosage (e.g., the second dosage plus about 50 to about 200 mg) for 3, 4, 5, 6, or 7 days, then proceeding to step (4);

(4) repeating the steps above through a fourth dosage, fifth dosage, etc., until no ER binding activity is detected.

In certain embodiments, the invention includes the use of PET imaging to detect and/or dose ER sensitive or ER resistant cancers.

### Combinations for the Methods Disclosed Herein

Another aspect of the invention relates to a pharmaceutical composition comprising RAD1901 or solvates (e.g., hydrate) or salts thereof and/or abemaciclib in a therapeutically effective amount as disclosed herein for the combination methods set forth herein.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

In order that the invention described herein may be more fully understood, the following examples are set forth. It should be understood that these examples are for illustrative purposes only and are not to be construed as limiting this invention in any manner.

### Example 1: An Experimental Clinical Trial Studying the Combination of Elacestrant (RAD1901) with Abemaciclib in Women with Advanced or Metastatic ER+/HER2− Breast Cancer

### Study Objectives:

### Primary

Safety Run-in: To determine the recommended Phase 2 dose (RP2D) of elacestrant in combination with abemaciclib in postmenopausal women with advanced or metastatic ER+/HER2− breast cancer who have not received prior therapy with a CDK4/6 inhibitor or a SERD.

Dose Expansion: To confirm the safety and tolerability of elacestrant in combination with abemaciclib at the selected RP2D in postmenopausal women with advanced or metastatic ER+/HER2− breast cancer whose disease has progressed on prior endocrine therapy.

### Secondary

To evaluate the clinical benefit rate (CBR)

To evaluate the objective response rate (ORR)

To evaluate the duration of response (DoR)

To evaluate progression free survival (PFS)

To assess the pharmacokinetic(s) (PK) of elacestrant in combination with abemaciclib

To assess the PK of abemaciclib in combination with elacestrant

### Exploratory

To evaluate genomic alterations relevant to ER+ breast cancer detected in circulating tumor DNA (ctDNA) and correlate with clinical response.

To evaluate biomarkers relevant to ER+ breast cancer in fresh and archival tumor biopsies and correlate with clinical response.

### Study Design:

This study is designed as a proof-of-concept to evaluate the safety and efficacy of elacestrant in combination with abemaciclib in postmenopausal women with advanced or metastatic ER+/HER2− breast cancer who have not received prior therapy with a CDK4/6 inhibitor or a SERD.

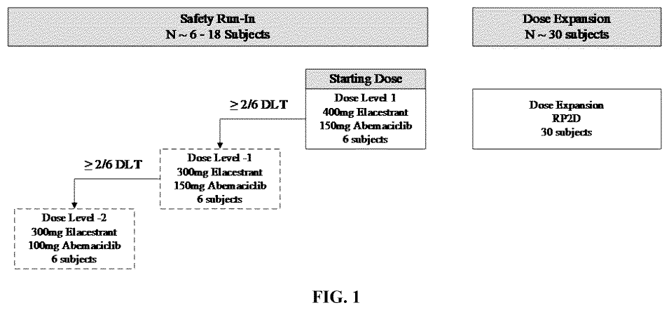

To evaluate the safety and tolerability of elacestrant in combination with abemaciclib, a Safety Run-in Phase will be performed to identify the maximum tolerated dose (MTD) and/or RP2D of the combination. Following completion of the Safety Run-In Phase, a Dose Expansion Phase will be opened to enroll 30 new subjects treated at the RP2D as provided in FIG. 1.

During the Safety Run-In phase, cohorts of six subjects will be enrolled sequentially at the Dose Levels provided in Table 1, beginning with Dose Level 1.

TABLE 1
Study Drug Dose Levels

| Dose Level | Elacestrant (mg, oral) | Abemaciclib (mg, oral) |
|---|---|---|
| −2 | 300 QD | 100 BID |
| −1 | 300 QD | 150 BID |
| 1 (starting dose) | 400 QD | 150 BID |

The initial cohort will evaluate Dose Level 1 and, based on safety review by the Study Committee and Sponsor, lower Dose Levels may be explored (Table 1). If needed, additional patients, intermediate doses or alternative dosing schedules may be explored to better define the safety, tolerability and PK of the elacestrant plus abemaciclib combination.

The MTD is defined as the highest dose at which 0/6 or 1/6 subjects or, if additional subjects are dosed, <33% of subjects at a dose level, experience a dose limiting toxicity (DLT) (Table 2) during the first 28 days of treatment. It is estimated that 2-3 dose levels will be required to determine an MTD and/or RP2D. The RP2D will be selected by the Study Committee and Sponsor based on evaluation of safety, PK and preliminary efficacy data. In the Dose Expansion Phase of the study, 30 new subjects will be enrolled to further evaluate tolerability and efficacy of the drug combination at the selected RP2D.

Elacestrant will be administered orally once daily at 400 or 300 mg on a continuous dosing schedule. Abemaciclib will be concurrently administered orally twice daily at 150 or 100 mg on a continuous daily schedule.

TABLE 2
Criteria for Dose Limiting Toxicities

| System | Criteria for Dose Limiting Toxicity (DLT) |
|---|---|
| Hematology | CTCAE Grade ≥3 anemia persists |
| | for >14 days despite RBC |
| | Febrile neutropenia (decrease in |
| | neutrophils associated with |
| | fever ≥38.5° C., ANC <1.0 × 109/L) |
| | with Grade 4 neutropenia |
| | CTCAE Grade 4 neutropenia for |
| | more than 14 consecutive days |
| | CTCAE Grade 3 thrombocytopenia |
| | with clinically significant |
| | bleeding |
| | CTCAE Grade 4 thrombocytopenia |
| Gastrointestinal | ≥ CTCAE Grade 4 or Grade 3 nausea |
| | >72 hours despite optimal* |
| | anti-emetic therapy |
| | ≥ CTCAE Grade 4 or Grade 3 |
| | dyspepsia that persists for >7 days |
| | despite optimal* therapy |
| | ≥ CTCAE Grade 4 or Grade 3 |
| | vomiting ≥72 hours despite optimal* |
| | anti-emetic therapy |
| | ≥ CTCAE Grade 4 or Grade 3 |
| | diarrhea ≥72 hours despite optimal* |
| | anti-diarrhea treatment |
| Hepatobiliary | CTCAE Grade 2 bilirubin >7 |
| | consecutive days |
| | ≥ CTCAE Grade 3 total bilirubin |
| | ≥ CTCAE Grade 2 ALT with a ≥ Grade |
| | 2 bilirubin elevation of any |
| | duration |
| | CTCAE Grade 3 ALT >7 consecutive days |
| | CTCAE Grade 4 ALT or AST |
| Cardiac | QTcF interval ≥501 ms on at least |
| | two separate electrocardiograms |
| | (ECGs) |
| Renal | ≥ CTCAE Grade 3 serum creatinine |
| | or Grade 2 for >7 days |
| Additional | ≥ CTCAE Grade 3 possibly related |
| Non- | to study drug(s), except for the |
| hematologic | exclusions noted below: |
| events | CTCAE Grade 3 fatigue <5 consecutive days |
| | CTCAE Grade 3 infection persists ≤7 days |
| | CTCAE Grade 3 fever in the |
| | absence of Grade 3/4 neutropenia |
| | persists ≤7 days with adequate antipyretic therapy |
| | CTCAE Grade 3 rash persists ≤7 days |
| All Events | AEs related to study drug(s) that lead |
| | to dose delay of >14 days prior |
| | to the scheduled start date of Cycle 2. |
| | AEs related to study drug(s) leading |
| | to drug interruption that prevents |
| | administration of either 75% of the |
| | dose of abemaciclib or 50% of |
| | doses of both abemaciclib and |
| | elacestrant together (ie, same day) |
| | during the first cycle (except Grade 3 |
| | neutropenia). |

AE = adverse event; ALT = alanine aminotransferase; ANC = absolute neutrophil count; CTCAE = Common Terminology Criteria for Adverse Events; RBC = red blood cell. CTCAE version 5.0 will be used for all grading.
*Optimal therapy for vomiting will be based on institutional guidelines, with consideration of the prohibited medications listed in this protocol. Optimal therapy for diarrhea will be based on abemaciclib prescribing information: see Table 4.

### Dose Adjustments and Dosing Delays

Every effort will be made to administer study drugs at the planned dose and schedule. However, dose adjustments or delays are permitted, as described in Table 3, Table 4, Table 5 and Table 6, in the event of significant treatment-related toxicity. Subjects requiring more than two dose reductions of both study drugs will be discontinued from the study.

Should drug-related toxicity mandate interruption of treatment with the suspect drug(s), subjects may continue to receive the other drug alone. If suspect drug-related toxicities have not resolved to the extent that a new cycle of combination therapy can begin on the scheduled Day 1 of the next cycle, the other drug may be continued and the suspect drug adjusted until toxicity resolves as described in Table 3, Table 4, Table 5 and Table 6. If abemaciclib-related toxicity mandates abemaciclib discontinuation, subjects may continue to receive elacestrant alone until progressive disease (PD), symptomatic deterioration, unacceptable toxicity, death, or withdrawal of consent, whichever occurs first. If elacestrant-related toxicity mandates elacestrant discontinuation, subjects may continue to receive abemaciclib alone until progressive disease (PD), symptomatic deterioration, unacceptable toxicity, death, or withdrawal of consent, whichever occurs first. If toxicities related to both drugs have not resolved to the extent that a new cycle of combination therapy can begin on the scheduled Day 1 of the next cycle, dosing can be delayed until toxicity resolves as described in Table 3, Table 4, Table 5 and Table 6.

TABLE 3
Dose Adjustment and Management-Hematologic Adverse Reactions
Monitor complete blood counts (CBCs) prior to the start of abemaciclib
therapy and at the beginning of each cycle, as well as on Day 15 of
the first two cycles, and as clinically indicated.

| | Abemaciclib Dose | Elacestrant Dose |
|---|---|---|
| CTCAE Grade | Adjustment | Adjustment |
| Grade 1 or 2 | No dose adjustment | No dose adjustment |
| | is required | is required |
| Grade 3 | Day 1 of a cycle: | No dose adjustment |
| | Withhold abemaciclib, repeat | is required |
| | CBC monitoring within 1 | |
| | week. When recovered to | |
| | Grade ≤2, start the next | |
| | cycle at the same dose. | |
| | Day 15 of first two cycles: | |
| | Continue abemaciclib at | |
| | current dose to complete | |
| | cycle. Repeat CBC on Day | |
| | 21. | |
| | Dose reduction is not required | |
| Grade 3 recurrent | Withhold abemaciclib until | No dose adjustment |
| events | recovery to Grade ≤2. | is required |
| | Resume at next lower dose. | |
| | Any episodes of fever should | |
| | be promptly reported | |
| Grade 4 | Withhold abemaciclib until | No dose adjustment |
| | recovery to Grade ≤2. | is required |
| | Resume at next lower dose. | |
| Patient requires | Suspend abemaciclib dose for | No dose adjustment |
| administration of | at least 48 hours after the last | is required |
| blood cell growth | dose of blood cell growth | |
| factors | factors was administered and | |
| | until toxicity resolves to | |
| | Grade 2 or less. Resume at | |
| | next lower dose unless the | |
| | dose was already reduced for | |
| | the toxicity that led to the | |
| | use of the growth factor. | |

CTCAE = Common Terminology Criteria for Adverse Events.
Grading according to CTCAE 5.0
Absolute neutrophil count (ANC): Grade 1: ANC < LLN-1500/μL; Grade 2: ANC <1500-1000/μL; Grade 3: ANC <1000-500/μL; Grade 4: ANC <500/μL

TABLE 4
Dose Adjustment and Management-Adverse Reactions of Diarrhea
At the first sign of loose stools start antidiarrheal therapy with
agents such as loperamide, increase oral fluids, and
notify healthcare provider.

| | Abemaciclib Dose | Elacestrant Dose |
|---|---|---|
| CTCAE Grade | Adjustment | Adjustment |
| Grade 1 | No dose adjustment is | No dose adjustment |
| | required | is required |
| Grade 2 | If toxicity does not | No dose adjustment |
| | resolve within 24 | is required |
| | hours to ≤ Grade 1, | |
| | suspend dose until | |
| | resolution. No dose | |
| | reduction is required. | |
| Grade 2 that persists or | Suspend dose until | No dose adjustment |
| recurs after resuming the | toxicity resolves | is required |
| same dose despite maximal | to ≤ Grade 1. Resume | |
| supportive measures | at next lower dose. | |
| Grade 3 or 4 or requires | Suspend dose until | No dose adjustment |
| hospitalization | toxicity resolves | is required |
| | to ≤ Grade 1. Resume | |
| | at next lower dose. | |

TABLE 5
Dose Adjustment and Management-Hepatotoxic Adverse Reactions
Monitor ALT, AST, and serum bilirubin prior to the start of
abemaciclib therapy, every 2 weeks for the first 2 months,
monthly for the next 2 months, and as clinically indicated.

| | Abemaciclib Dose | Elacestrant Dose |
|---|---|---|
| CTCAE Grade | Adjustment | Adjustment |
| Grade 1 (>ULN-3.0 × ULN) | No dose adjustment is | No dose |
| Grade 2 (>3.0-5.0 × ULN), | required | adjustment |
| WITHOUT increase in total | | is required |
| bilirubin above 2 × ULN | | |
| Persistent or Recurrent | Suspend dose | No dose |
| Grade 2, or Grade 3 (>5.0 | until toxicity | adjustment |
| 20.0 × ULN), WITHOUT | resolves to baseline or | is required |
| increase in total bilirubin | Grade 1. Resume at | |
| above 2 × ULN | next lower dose. | |
| Elevation in AST and/or | Discontinue | No dose |
| ALT >3 × ULN WITH total | abemaciclib | adjustment |
| bilirubin >2 × ULN, | | is required |
| in the absence of | | |
| cholestasis | | |
| Grade 4 (>20.0 × ULN) | Discontinue | No dose |
| | abemaciclib | adjustment |
| | | is required |

ALT = alanine aminotransferase, AST = aspartate aminotransferase, ULN = upper limit of normal.

TABLE 6
Dose Adjustment and Management-Non-Hematologic
Adverse Reactions

| CTCAE Grade | Dose Adjustment |
|---|---|
| Grade 1 or 2 | No dose adjustment is required for |
| | either abemaciclib or elacestrant |
| Persistent or | If adverse reaction is related to either |
| recurrent Grade 2 | abemaciclib or elacestrant, or to both drugs: |
| toxicity that does | Withhold suspect drug(s) until symptoms |
| not resolve with | resolve to Grade ≤1 |
| maximal supportive | Resume study drug(s) at the next lower dose. |
| measures within 7 | NOTE: If symptoms do not recur after |
| days to baseline or | re-challenge at a lower dose, dose |
| Grade 1 | re-escalation may be considered following |
| | discussion with the Sponsor. |
| Grade ≥3 | If adverse reaction is related to either |
| non-hematologic | abemaciclib or elacestrant, or to both drugs: |
| toxicity (if | Withhold suspect drug(s) until symptoms |
| persisting >48 | resolve to Grade ≤1 |
| hours despite | Resume both study drugs at the next lower dose. |
| optimal medical | Resume both study drugs at the next lower dose. |
| treatment) | NOTE: If symptoms do not recur |
| | after re-challenge at a lower dose, |
| | dose re-escalation may be considered |
| | following discussion with the Sponsor. |

CTCAE = Common Terminology Criteria for Adverse Events.
Grading according to CTCAE 5.0

### Subject Population:

Postmenopausal women with advanced or metastatic ER+/HER2− breast cancer whose disease progressed on prior AI therapy.

### Inclusion and Exclusion Criteria:

Subjects must meet all of the following inclusion and none of the exclusion criteria:

### Inclusion Criteria

Subjects with histologically or cytologically proven diagnosis of adenocarcinoma of the breast with evidence of recurrent (either locally or metastatic) disease.
Subjects must have measurable and/or evaluable disease per Response Evaluation Criteria in Solid Tumors (RECIST v1.1). Tumor lesions previously subjected to radiation therapy or other locoregional therapy will be considered measurable and/or evaluable only if disease progression after completion of locoregional therapy is clearly documented. Bone lesions or mixed lytic-blastic lesions that can be evaluated by cross-sectional imaging techniques such as CT or MRI can be considered evaluable lesions if they meet the definition of evaluable disease as defined by RECIST v1.1. Blastic bone lesions are evaluable lesions.
Subjects must be postmenopausal women, defined as:
   a. Documented bilateral surgical oophorectomy
   b. Age ≥60 years with amenorrhea ≥1 year since last menses
   c. Age <60 years with amenorrhea ≥1 year since last menses with no alternative pathological or physiological cause (including chemotherapy, treatment with tamoxifen or toremifene, or a GnRH agonist), and serum estradiol and FSH level within the laboratory's reference range for postmenopausal women.
Age ≥18 years.
Subjects must have the following tumor status confirmed per local laboratory testing on their most recent biopsy from the primary tumor or metastatic lesion:
   a. ER+ tumor with ≥1% staining by IHC as defined in the 2010 American Society of Clinical Oncology (ASCO) recommendations for ER testing
   b. HER2− tumor with an IHC result of 0 or 1+ for cellular membrane protein expression or an in situ hybridization (ISH) negative result as defined in the 2013 ASCO recommendations for HER2 testing
Subjects may have received no more than 2 lines of prior endocrine therapy for advanced or metastatic disease, not including a CDK4/6 inhibitor or a SERD, and must have documented evidence of newly metastatic disease, or of progression of previously treated metastatic disease.
Subjects may have received one prior chemotherapeutic regimen in the advanced/metastatic setting (prior adjuvant chemotherapy is permitted if it was ≥12 months before enrollment). Chemotherapy administered for less than one cycle will not be counted as a prior line of therapy.
Eastern Cooperative Oncology Group (ECOG) performance status 0 or 1.
Resolution of all toxic effects of prior therapies or surgical procedures to Grade≤1 (except alopecia and peripheral neuropathy).
Adequate organ function as defined below:
   a. Hematologic function
      i. Absolute neutrophil count (ANC) ≥1500/μL
      ii. Platelet count ≥100,000/μL
      iii. Hemoglobin ≥8.0 gm/dL. Patients may receive erythrocyte transfusions to achieve this hemoglobin level at the discretion of the investigator; however, initial study drug treatment must not begin earlier than the day after the erythrocyte transfusion.
   b. Renal function
      i. Calculated serum creatinine >30 mL/min calculated by the Cockcroft-Gault formula
   c. Hepatic function
      i. Alanine aminotransferase (ALT)≤3x upper limit of normal (ULN)
      ii. Aspartate aminotransferase (AST)≤3×ULN
      iii. Total bilirubin≤ULN, or total bilirubin≤1.5×ULN with direct bilirubin≤ULN in subjects with documented Gilbert's Syndrome.
   d. Chemistry
      i. Potassium, sodium, calcium (corrected for albumin), magnesium, and phosphorus within the normal range of the laboratory. If screening assessments are abnormal, chemistry assessments may be repeated up to two times; subjects may receive appropriate supplementation prior to re-assessment
   e. Coagulation
      i. INR≤1.5
         Note: Subjects who are receiving anticoagulation treatment may be allowed to participate with a stable INR established within the therapeutic range for at least one month prior to the first dose of study drug, in the absence of any exclusionary medical conditions, and provided that an AI would be an appropriate therapy for the subject.
Ability to read, understand, and sign an informed consent document

### Exclusion Criteria

Prior treatment with fulvestrant or a CDK4/6 inhibitor.
Prior treatment with elacestrant (RAD1901), GDC-0810, GDC-0927, GDC-9545, LSZ102, AZD9496, bazedoxifene, or other investigational SERD or ER antagonist.
Prior anti-cancer or investigational drug treatment within the following windows:
   a. Any endocrine therapy less than 14 days before first dose of study treatment
   b. Any chemotherapy less than 21 days before first dose of study treatment
   c. Any investigational anti-cancer drug therapy less than 21 days or three half-lives (whichever is longer) before the first dose of study treatment
Presence of symptomatic metastatic visceral disease, defined as extensive hepatic involvement, untreated or progressive CNS metastases, or symptomatic pulmonary lymphangitic spread. Subjects with discrete pulmonary parenchymal metastases are eligible, provided their respiratory function is not significantly compromised as a result of disease in the opinion of the Investigator. Subjects with previously treated CNS metastases are eligible provided that all known lesions were previously treated, they have completed radiotherapy at least 28 days prior to first dose of study drug, are clinically stable, and require no steroid medication. If anti-convulsant medication is required, subjects must be stable on a non-enzyme inducing anticonvulsant regimen.
Subjects with an intact uterus with a history of endometrial intraepithelial neoplasia (atypical endometrial hyperplasia or higher-grade lesion).
Diagnosis of any other malignancy within 3 years prior to enrollment, except for adequately treated basal cell or squamous cell skin cancer, or carcinoma in situ of the cervix.
Any of the following within six months before enrollment: myocardial infarction, severe/unstable angina, ongoing cardiac dysrhythmias≥Grade 2, prolonged QTcF≥Grade 2, uncontrolled atrial fibrillation of any grade, coronary/peripheral artery bypass graft, heart failure≥Class II as defined by the New York Heart Association (NYHA) guidelines, or cerebrovascular accident including transient ischemic attack or symptomatic pulmonary embolism.
Subjects with abnormal coagulation profiles or a history of coagulopathy within the past 6 months, including history of deep vein thrombosis (DVT) or pulmonary embolism. Subjects with the following conditions will be allowed to participate:
   a. Subjects with adequately treated catheter-related venous thrombosis occurring more than one month prior to the first dose of study drug.
   b. Subjects being treated with anticoagulant, eg, warfarin or heparin, for a thrombotic event occurring more than 6 months before enrollment, or for an otherwise stable and allowed medical condition (e.g., well controlled atrial fibrillation), provided dose and coagulation parameters (as defined by local standard of care) are stable for at least one month prior to the first dose of study treatment.
Subjects with known difficulty in swallowing oral medications, or who have had a diagnosis of any of the following: severe diarrhea, uncontrolled nausea or vomiting, gastrointestinal (GI) obstruction/motility disorder, malabsorption syndrome, or gastric bypass.
Subjects who are receiving treatment with medications, or consuming herbal supplements, and/or fruits (eg, pomelos, star fruit, Seville oranges) that are known to be strong inhibitors or inducers of CYP3A4 that cannot be discontinued within five half-lives or 14 days, whichever is longer, before study entry and for the duration of the study.
Major surgery within 28 days before the first dose of study treatment.
Radiation therapy within 14 days before the first dose of study treatment. Radiated lesions should not be selected as target lesions.
Any concurrent severe, acute, or chronic medical or psychiatric condition or laboratory abnormality that may increase the risk associated with study participation or investigational product administration or may interfere with the interpretation of study results and, in the judgment of the Investigator, would make the individual inappropriate for entry into this study.

### Duration of Therapy:

Subjects will continue to receive treatment until confirmed PD, unacceptable toxicity, death, or withdrawal of consent, whichever occurs first.

### Duration of Follow-Up:

All subjects will be followed until 30 days post-treatment or until resolution or stabilization of all treatment-related AEs to Grade 2 or lower.

### Duration of Study Conduct:

Approximately 36 months or until the last subject has completed study treatment and the 30-day follow-up period.

### Investigational Product, Dose, and Mode of Administration:

Elacestrant will be supplied as 100 or 400 mg tablets and will be administered orally daily on a continuous dosing schedule. The starting dose for the Safety Run-in Phase will be 400 mg.

Abemaciclib will be supplied as 100 or 150 mg tablets and will be administered twice daily on a continuous daily dosing schedule. The starting dose for the Safety Run-in Phase will be 150 mg.

### Study Endpoints:

### Primary

Safety Run-in: Frequency of DLTs during the first 28 days of treatment with elacestrant in combination with abemaciclib treatment.

Dose Expansion: Incidence of all adverse events (AEs), all serious adverse events (SAEs), review of laboratory data (including hematology and chemistry), ECG monitoring, physical examinations, performance status, and vital signs.

### Secondary

CBR is defined as the proportion of subjects with a best overall response of complete response (CR), partial response (PR), or stable disease (SD)≥24 weeks. Tumor response will be determined by the Investigator according to RECIST v1.1 guidelines.

ORR is defined as the proportion of subjects with a best overall response of CR or PR. Tumor response will be determined by the Investigator according to RECIST v1.1.

DoR is calculated as the time from the date of first documented response (CR or PR) to the first documented date of tumor progression. Tumor response and progression will be determined by the Investigator according to RECIST v1.1.

PFS is calculated as the length of time from the date of first dose until the earlier date of documented disease progression per RECIST v1.1 or death from any cause.

Pharmacokinetic parameters will include area under the concentration-time curve (AUC), time of maximum concentration (tmax), maximum plasma concentration (Cmax), oral clearance (CL/F), and other PK parameters as appropriate.

### Exploratory

Serially collected blood samples will be analyzed for genomic alterations relevant to ER+ breast cancer in the circulating tumor DNA (ctDNA) Tumor biopsies will be analyzed for biomarkers relevant to ER+ breast cancer.

### Number of Subjects:

Up to 48 subjects will be enrolled, with up to 36 subjects treated at the RP2D. This assumes up to three Dose Levels in the Safety Run-in Phase with cohorts of 6 subjects each (6-18 subjects) and an additional 30 subjects enrolled during the Dose Expansion Phase. Subjects who discontinue treatment before completing the Safety Run-in Phase (Day 1 to 28) for reasons other than toxicity may be replaced.

### Sample Size Assumptions:

The sample size in the Safety Run-in Phase is customary for a dose escalation study. The Safety Run-in Phase will evaluate up to three Dose Levels, in cohorts of 6 subjects each; the total sample size is expected to be 6-18 subjects depending on the number of cohorts.

A total of approximately 30 new subjects will be enrolled in the Dose Expansion Phase. With a combined ˜36 subjects treated at the RP2D (30 from the Dose Expansion Phase plus approximately six subjects from the Safety Run-in Phase), the study will have a greater than 90% chance to detect an AE with an incidence rate of 7% or higher.

Although not the primary objective, the Dose Expansion Phase will be used to generate preliminary efficacy data for the combination treatment. Assuming a CBR at 24 weeks of 75%, a total of 32 evaluable subjects (36 subjects minus 10% drop out) will have a 95% lower confidence limit of 58% based on the Wilson method.

The primary data analysis will occur approximately 18 months after the last subject is enrolled. Subjects will continue to be followed up for objective disease progression until approximately 50% of subjects have died or experienced objective disease progression, at which time the final analysis will be conducted.

### Example 2: An Experimental Clinical Trial Studying the Combination of

### Elacestrant with Abemaciclib for Treatment of ER+/HER2− Advanced Breast Cancer (Vs Investigator Choice of Nonsteroidal Aromatase Inhibitor+Abemaciclib or Fulvestrant+Abemaciclib)

### Study Overview

This is an international, multicenter, randomized, active-controlled, open-label Phase 3 clinical trial comparing the efficacy and safety of elacestrant in combination with abemaciclib versus letrozole or fulvestrant in combination with abemaciclib in subjects with ER+/HER2− advanced/metastatic breast cancer who have progressed on or after prior adjuvant or metastatic endocrine therapy, and have not received prior treatment with a CDK4/6 inhibitor or a SERD. The primary objective is to demonstrate that the combination of elacestrant plus abemaciclib is superior to 1) a combination of letrozole and abemaciclib or 2) a combination of anastrozole and abemaciclib or 3) a combination of fulvestrant and abemaciclib in prolonging PFS.

Approximately 600 (HR<0.7) OR 1000 (HR<0.8) subjects will be randomized 1:1 to receive either:
a. Elacestrant+Abemaciclib
b. Investigator choice of nonsteroidal aromatase inhibitors (AI) (Letrozole/Anastrozole)+Abemaciclib or Fulvestrant+Abemaciclib
Elacestrant: dose TBD (up to 400 mg), orally once daily on a continuous dosing schedule.
Abemaciclib: 125 mg, twice daily on a continuous dosing schedule.
Letrozole: 2.5 mg, orally once daily on a continuous doing schedule.
Anastrozole: 1 mg, orally once daily.
Fulvestrant: 500 mg, per label.

### Endpoints:

### Primary Endpoint

a. Progression Free Survival (PFS)

### Secondary Endpoints

a. Overall Survival OS
b. Objective Response Rate (ORR)
c. Duration of Response (DoR)
d. Clinical Benefit Rate (CBR)
e. Safety and tolerability
f. Pharmacokinetics (PK)
g. Quality of Life (QoL)

### Example 3: An Experimental Clinical Trial Studying the Combination of Elacestrant with Abemaciclib for First Line Treatment of ER+/HER2− Advanced Breast Cancer

### Study Overview

This is an international, multicenter, randomized, active-controlled, open-label Phase 3 clinical trial comparing the efficacy and safety of elacestrant in combination with abemaciclib versus letrozole in combination with abemaciclib in subjects with ER+/HER2− advanced/metastatic breast cancer who have not received prior systemic anti-cancer therapies for their advanced/metastatic disease and have not received prior treatment with a CDK4/6 inhibitor or a SERD. The primary objective is to demonstrate that the combination of elacestrant plus abemaciclib is superior to letrozole plus abemaciclib in prolonging PFS.

Approximately 650 (HR<0.7) OR 1100 (HR<0.8) subjects will be randomized 1:1 to receive either:
a. Elacestrant+Abemaciclib
b. Letrozole+Abemaciclib
Elacestrant: dose TBD (up to 400 mg), orally once daily on a continuous dosing schedule.
Abemaciclib: 125 mg, twice daily on a continuous dosing schedule.
Letrozole: 2.5 mg, orally once daily on a continuous doing schedule.

### Endpoints:

### Primary Endpoint

a. Progression Free Survival (PFS)

### Secondary Endpoints

a. Overall Survival OS
b. Objective Response Rate (ORR)
c. Duration of Response (DoR)
d. Clinical Benefit Rate (CBR)
e. Safety and tolerability
f. Pharmacokinetics (PK)
g. Quality of Life (QoL)

### Example 4: An Experimental Clinical Trial Studying the Combination of Elacestrant with Abemaciclib Vs Abemaciclib Alone for the Treatment of ER+/HER2− Advanced Breast Cancer

### Study Overview

This is an international, multicenter, randomized, active-controlled, double-blind Phase 3 clinical trial comparing the efficacy and safety of elacestrant in combination with abemaciclib versus abemaciclib alone in subjects with ER+/HER2− advanced/metastatic breast cancer who have received prior systemic anti-cancer therapy including ≤2 prior chemotherapy for mBC permitted for their advanced/metastatic disease, not including a CDK4/6 inhibitor or a SERD. The primary objective is to demonstrate that the combination of elacestrant plus abemaciclib is superior to abemaciclib alone in prolonging PFS. Approximately 500 subjects (HR<0.7) will be randomized 2:1 to receive either:
a. Elacestrant+Abemaciclib
b. Abemaciclib
Elacestrant: dose TBD (up to 400 mg), orally once daily on a continuous dosing schedule.
Abemaciclib: 150 mg, twice daily on a continuous daily schedule in combination or 200 mg taken orally twice daily as a monotherapy.

### Endpoints:

### Primary Endpoint

a. Progression Free Survival (PFS)

### Secondary Endpoints

a. Overall Survival OS
b. Objective Response Rate (ORR)
c. Duration of Response (DoR)
d. Clinical Benefit Rate (CBR)
e. Safety and tolerability
f. Pharmacokinetics (PK)
g. Quality of Life (QoL)

### Other Embodiments

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms used in this disclosure, the meaning of the terms in this disclosure are intended to be controlling. Furthermore, the foregoing discussion discloses and describes merely exemplary embodiments of the present invention. One skilled in the art will readily recognize from such discussion and from the accompanying drawings and claims, that various changes, modifications and variations can be made therein without departing from the spirit and scope of the invention as defined in the following claims.

What is claimed is:

1. A method of treating breast cancer in a patient, comprising administering to the patient a therapeutic combination comprising elacestrant, or a pharmaceutically acceptable salt thereof, and abemaciclib, or a pharmaceutically acceptable salt thereof.

2. The method of claim 1, wherein the patient is a postmenopausal women.

3. The method of claim 1, wherein the patient has not received prior therapy with a CDK4/6 inhibitor or a SERD.

4. The method of claim 1, wherein the breast cancer in the patient has progressed on prior endocrine therapy.

5. The method of claim 1, wherein the breast cancer in the patient is ER+ breast cancer.

6. The method of claim 5, wherein the breast cancer in the patient is HER2− breast cancer.

7. The method of claim 1, wherein the breast cancer in the patient is advanced or metastatic breast cancer.

8. The method of claim 1, wherein the elacestrant is administered to the patient at a dose of from 200-500 mg/day.

9. The method of claim 8, wherein the elacestrant is administered to the patient at a dose of from 250-450 mg/day.

10. The method of claim 9, wherein the elacestrant is administered to the patient at a dose of about 300 mg/day.

11. The method of claim 10, wherein the elacestrant is administered to the patient at a dose of about 300 mg/day, in one administration per day.

12. The method of claim 9, wherein the elacestrant is administered to the patient at a dose of about 400 mg/day.

13. The method of claim 12, wherein the elacestrant is administered to the patient at a dose of about 400 mg/day, in one administration per day.

14. The method of claim 1, wherein the abemaciclib is administered to the patient at a dose of from 150-400 mg/day.

15. The method of claim 14, wherein the abemaciclib is administered to the patient at a dose of about 200 mg/day.

16. The method of claim 15, wherein the abemaciclib is administered to the patient at a dose of about 200 mg/day, in two administrations per day.

17. The method of claim 16, wherein the abemaciclib is administered to the patient at a dose of about 100 mg twice per day.

18. The method of claim 14, wherein the abemaciclib is administered to the patient at a dose of about 300 mg/day.

19. The method of claim 18, wherein the abemaciclib is administered to the patient at a dose of about 300 mg/day, in two administrations per day.

20. The method of claim 19, wherein the abemaciclib is administered to the patient at a dose of about 150 mg twice per day.

21. The method of claim 1, wherein the elacestrant is administered to the patient at a dose of about 400 mg/day, and the abemaciclib is administered to the patient at a dose of about 300 mg/day.

22. The method of claim 21, wherein the abemaciclib is administered to the patient at a dose of 150 mg twice per day.

23. The method of claim 1, wherein the elacestrant is administered to the patient at a dose of about 300 mg/day, and the abemaciclib is administered to the patient at a dose of about 300 mg/day.

24. The method of claim 23, wherein the abemaciclib is administered to the patient at a dose of 150 mg twice per day.

25. The method of claim 1, wherein the elacestrant is administered to the patient at a dose of about 300 mg/day, and the abemaciclib is administered to the patient at a dose of about 200 mg/day.

26. The method of claim 25, wherein the abemaciclib is administered to the patient at a dose of 100 mg twice per day.

27. The method of claim 1, wherein the elacestrant is administered to the patient at a dose that is the maximum tolerated dose for the patient.

28. The method of claim 1, wherein the abemaciclib is administered to the patient at a dose that is the maximum tolerated dose for the patient.

29. The method of claim 1, wherein the patient experiences a greater Progression Free Survival time as compared to a patient who was administered a combination of letrozole and abemaciclib, a combination of anastrozole and abemaciclib, or a combination of fulvestrant and abemaciclib.

30. The method of claim 29, wherein the breast cancer is ER+/HER2− advanced or metastatic breast cancer, and the patient has progressed on or after prior adjuvant or metastatic endocrine therapy, and has not received prior treatment with a CDK4/6 inhibitor or a SERD.

31. The method of claim 29, wherein the patient who was administered a combination of letrozole and abemaciclib was administered 2.5 mg once per day of letrozole and 125 mg twice daily of abemaciclib.

32. The method of claim 29, wherein the patient who was administered a combination of anastrozole and abemaciclib was administered 1 mg once per day of anastrozole and 125 mg twice daily of abemaciclib.

33. The method of claim 29, wherein the patient who was administered a combination of fulvestrant and abemaciclib was administered 500 mg fulvestrant injection as two 5 mL injections intramuscularly into the buttocks (gluteal area), at a rate of 1-2 minutes per injection, one in each buttock, on days 1, 15, and 29 and once monthly thereafter, and 125 mg twice daily of abemaciclib.

34. The method of claim 29, wherein the patient experiences a greater Progression Free Survival time as compared to a patient who was administered a combination of letrozole and abemaciclib and wherein the breast cancer is ER+/HER2− advanced or metastatic breast cancer, and the patient has not received prior systemic anti-cancer therapies for their advanced/metastatic disease, and has not received prior treatment with a CDK4/6 inhibitor or a SERD.

35. The method of claim 34, wherein the patient who was administered a combination of letrozole and abemaciclib was administered 2.5 mg once per day of letrozole and 125 mg twice daily of abemaciclib.

36. The method of claim 1, wherein the patient experiences a greater Progression Free Survival time as compared to a patient who was administered abemaciclib as a monotherapy.

37. The method of claim 36, wherein the breast cancer is ER+/HER2− advanced or metastatic breast cancer, and the patient has received prior systemic anti-cancer therapy including ≤2 prior chemotherapy for metastatic breast cancer permitted for their advanced or metastatic disease, and wherein the prior systemic anti-cancer therapy did not include a CDK4/6 inhibitor or a SERD.

38. The method of claim 36, wherein the patient who was administered abemaciclib as a monotherapy was administered 200 mg of abemaciclib twice daily.
